(19) United States
(12) Patent Application Publication
(10) Pub. No.: US 2010/0186763 A1
(43) Pub. Date: Jul. 29, 2010

(54) COSMETIC COMPOSITION COMPRISING AT LEAST ONE LAMBDA-CARRAGEENAN POLYSACCHARIDE AND INORGANIC PARTICLES

(76) Inventors: Ludivine LAURENT, Chartres (FR); Valérie Vieira, Pomponne (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/626,135

(22) Filed: Nov. 25, 2009

Related U.S. Application Data
(60) Provisional application No. 61/149,466, filed on Feb. 3, 2009.

(30) Foreign Application Priority Data
Nov. 28, 2008 (FR) 0858099

(51) Int. Cl. A61K 8/73; A61Q 5/06

(52) U.S. Cl. 132/202; 514/54; 424/70.13

(57) ABSTRACT

The present disclosure relates to a cosmetic composition comprising, in a cosmetically acceptable medium, at least one lambda-carrageenan polysaccharide and inorganic particles chosen from the particles comprising at least 10% by weight of at least one silicate, to a cosmetic treatment method employing the cosmetic composition and to the use of this composition for styling the hair.

This application claims benefit of U.S. Provisional Application No. 61/149,466, filed Feb. 3, 2009, the contents of which are incorporated herein by reference. This application also claims benefit of priority under 35 U.S.C. §119 to French Patent Application No. FR 0858099, filed Nov. 28, 2008, the contents of which are also incorporated herein by reference.

The present disclosure relates to a cosmetic composition comprising, in a cosmetically acceptable medium, at least one lambda-carageenan polysaccharide and specific inorganic particles, and to a method for the cosmetic treatment of keratinous fibers and to the use of the cosmetic composition for styling the hair.

In the styling field, for example among hair products intended for shaping and/or retaining the hairstyle, hair compositions are generally composed of a solution, generally alcoholic or aqueous, and of at least one fixing polymer as a mixture with various cosmetic adjuvants.

These compositions can be provided in the form of hair gels or mousses which are generally applied to wet hair before blow drying or drying.

For example, hair gels may be composed of at least one thickening polymer or gelling agent in combination with at least one fixing polymer which generally has the role of forming a film at the surface of the keratinous fibers to be fixed.

The use of carrageenans as fixing polymers in styling gels or in aerosols is known. For example, the use of at least one lambda-carageenan polysaccharide may make it possible to obtain styling products exhibiting good styling and cosmetic properties.

Carrageenans are polysaccharides which form the cell walls of various red algae (Rhodophyceae) belonging to the families of the Gigartinaceae, Hypneaceae, Furcellariaceae and Polyideaceae. They comprise long galactan chains which are anionic polyelectrolytes. Their molecular weights can, for example, be greater than 10⁶. These linear polymers, formed by disaccharide units, are composed of two D-galactopyranose units alternately bonded via α- and β-bonds. These are highly sulphated (20-50%) polysaccharides and the α-D-galactopyranosyl residues can be in the 3′,6′-anhydro form.

The carrageenans were initially subdivided into two families according to their solubility in potassium chloride (KCl). The fractions soluble in KCl were designated by the prefixes “kappa”, while the terms “lambda” were reserved for those which are insoluble. Later, the classifications were based on the number of sulphate groups, the position of sulphate groups and the presence of 3′,6′-anhydro bridge on the O-D-galactopyranosyl residues. This resulted in four main families: kappa, lambda, beta and omega.

The various types of carrageenans do not exist in the pure state but in the form of hybrids. Thus, in the natural state, the kappa- and lambda-carrageenans exist in a kappa-iota hybrid form but one of the two structures may predominate over the other. The kappa-lambda hybrid state of a structure can be elucidated using specific enzymes which may make it possible to enrich or reduce the content of one of the two forms. The carrageenans can coexist with their precursors. The carrageenans belonging to different families can coexist in a hybrid structure, for example: carrageenan from Euchema gelatinae, a hybrid of beta-carrageenan, major component, and of kappa- and gamma-carrageenans.

While the use of a lambda-carrageenan polysaccharide may make it possible to obtain styling products exhibiting good styling and cosmetic properties, the formation of visible and unsightly residues may be observed, however, on passing the comb through the hair.

There thus exists a real need to make available a cosmetic composition which may exhibit good styling and cosmetic properties and/or which may make it possible to overcome the abovementioned disadvantages.

In accordance with the disclosure, by combining at least one lambda-carrageenan polysaccharide with specific inorganic particles, it may be possible to obtain a hair composition which makes it possible to significantly reduce the appearance and the size of the residues when a comb is passed through the hair, while retaining good styling and cosmetic properties.

Accordingly, one aspect of the present disclosure is a cosmetic composition comprising, in a cosmetically acceptable medium:
at least one lambda-carrageenan polysaccharide, and
inorganic particles chosen from particles comprising at least 10% by weight of at least one silicate.

Another aspect of the disclosure is a cosmetic treatment method employing the cosmetic compositions according to the disclosure.

Another aspect of the disclosure is the use of the cosmetic compositions according to the disclosure for styling the hair.

Other aspects and benefits of the disclosure will become apparent on reading the description and examples which follow.

As used herein, the term “styling” is understood to mean the act of fixing and/or of retaining the form of the hairstyle.

The composition according to the disclosure comprises at least one lambda-carageenan polysaccharide.

The at least one lambda-carageenan polysaccharide may or may not be chemically modified. For example, in at least one embodiment the at least one lambda-carrageenan polysaccharide is not chemically modified.

For example, the molecular weight (MW) of the at least one lambda-carageenan polysaccharide may range from 100,000 to 1,000,000. For instance, the molecular weight may range from 250,000 to 800,000.

Mention may be made, as examples of lambda-carrageenan polysaccharides which may be present in the composition according to the disclosure, of SATIAGUM UTC 10 from Degussa and WELGEENAN ED 1039 from Eurogum.

The at least one lambda-carrageenan polysaccharide is, for example, present in a total amount ranging from 0.01 to 30% by weight relative to the total weight of the composition, such as from 0.1 to 20% by weight or for example from 1 to 10%.

The composition according to the disclosure also comprises inorganic particles chosen from particles comprising at least 10% by weight of at least one silicate.

The inorganic particles generally exhibit a number-average primary size, for example, ranging from 2 nm to 2 μm, such as from 5 nm to 500 nm, or such as from 10 nm to 250 nm.

The particles according to the disclosure can have any shape, for example the shape of spheres, flakes, needles or slabs, or completely random shapes. In at least one embodiment, they are substantially spherical.

As used herein, the term “particle primary size” is understood to mean the maximum dimension which it is possible to measure between two diametrically opposite points of an individual particle. The size can be determined, for example, by transmission electron microscopy or from the measurement of the specific surface area by the BET method or else via a laser particle sizer.

As explained above, the inorganic particles are particles comprising at least 10% by weight of at least one silicate. In other words, each of said inorganic particles comprises at least 10% by weight of at least one silicate.

In at least one embodiment, the particles comprising at least 10% by weight of at least one silicate may additionally comprise a metal or semi-metal oxide, for example silicon oxide, boron oxide or aluminium oxide.

In at least one embodiment, the inorganic particles are particles of at least one silicate, i.e., particles which substantially comprise only at least one silicate.

The silicates suitable in the compositions of the present disclosure can be of natural or synthetic origin.

The silicates which can be used according to the disclosure can be chosen from magnesium, lithium, and sodium silicates. Mention may, for example, be made of the compounds sold by Laporte under the names LAPONITE XLG and LAPONITE XLS.

The inorganic particles comprising at least 10% by weight of at least one silicate are generally present in an amount ranging from 0.01 to 20% by weight relative to the total weight of the composition, such as from 0.1 to 10% by weight or such as from 0.1 to 5% by weight, relative to the total weight of the composition.

The ratio of the total weight of the at least one lambda-carrageenan polysaccharide to the total weight of inorganic particles comprising at least 10% by weight of at least one silicate has a value that ranges from 0.1 to 10.

The cosmetic composition according to the disclosure may, for example, further comprise at least one additive chosen from silicones, fatty substances, polymers other than the at least one lambda-carrageenan polysaccharide, and surfactants.

The at least one silicone optionally present in the composition according to the disclosure may be, for example, polyorganosiloxanes which can be provided in the form of aqueous solutions, that is to say dissolved, or optionally in the form of aqueous emulsions, or dispersions or microdispersions. The polyorganosiloxanes can also, for example, be provided in the form of oils, waxes, resins or gums.

Organopolysiloxanes are defined in more detail in the work by Walter Noll, “Chemistry and Technology of Silicones”, (1968), Academic Press.

The silicones can be volatile or nonvolatile.

When they are volatile, the silicones may be chosen, for example, from those having a boiling point ranging from 60° C. to 260° C. and, for example:

(i) cyclic silicones comprising from 3 to 7 silicon atoms such as 4 or 5.

They include, for example, octamethylcyclotetrasiloxane, sold, for example, under the name of VOLATILE SILICONE 7207 by Union Carbide or SILBIONE 70045 V 2 by Rhône-Poulenc, decamethylcyclopentasiloxane, sold under the name of VOLATILE SILICONE 7158 by Union Carbide or SILBIONE 70045 V 5 by Rhône-Poulenc, and mixtures thereof.

Exemplary mention may also be made of cyclocopolymers of the dimethylsiloxane/methylalkylsiloxane type, such as SILICONE VOLATILE FZ 3109, sold by Union Carbide, with the chemical structure:

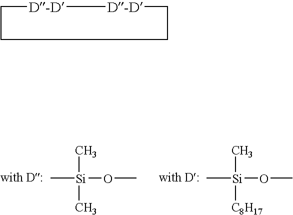

Exemplary mention may also be made of mixtures of cyclic silicones with silicon-derived organic compounds, such as the mixture of octamethylcyclotetrasiloxane and of tetratrimethylsilylpentaerythritol (50/50) and the mixture of octamethylcyclotetrasiloxane and of 1,1′-oxy(hexa-2,2,2′,2′,3,3′-trimethylsilyloxy)bisneopentane;

(ii) linear volatile silicones having from 2 to 9 silicon atoms and having a viscosity of less than or equal to 5×10⁻⁶ m²/s at 25° C. They include, for example, decamethyltetrasiloxane, sold, for example, under the name SH 200 by Toray Silicone. Silicones coming within this category are also described in the article published in Cosmetics and Toiletries, Vol. 91, Jan. 76, pp. 27-32, Todd & Byers, “Volatile Silicone Fluids for Cosmetics”.

Use may be made of nonvolatile silicones, for example of polyalkylsiloxanes, polyarylsiloxanes, polyalkylarylsiloxanes, silicone gums and resins, polyorganosiloxanes modified by organofunctional groups, and mixtures thereof.

In one embodiment, these silicones are chosen from polyalkylsiloxanes, among which exemplary mention may be made of polydimethylsiloxanes possessing trimethylsilyl end groups (Dimethicone according to the CTFA name) having a viscosity ranging from 5×10⁻⁶ to 2.5 m²/s at 25° C., such as from 1×10⁻⁵ to 1 m²/s. The viscosity of the silicones is, for example, measured at 25° C. according to Standard ASTM 445 Appendix C.

Mention may be made, among these polyalkylsiloxanes, for example, of the following commercial products:

SILBIONE oils of the 47 and 70 047 series or MIRASIL oils sold by Rhône-Poulenc, such as, for example, the oil 70 047 V 500 000;

oils of the MIRASIL series sold by Rhône-Poulenc; oils of the 200 series from Dow Corning, such as, for example, DC200 with a viscosity of 60,000 cSt;

VISCASIL oils from General Electric and some oils of the SF series (SF 96, SF 18) from General Electric.

Exemplary mention may also be made of polydimethylsiloxanes possessing dimethylsilanol end groups (Dimethiconol according to the CTFA name), such as oils of the 48 series from Rhône-Poulenc.

Mention may also be made, for example, of polydimethylsiloxanes possessing aminoethyl aminopropyl and α,ω-silanol groups.

Exemplary mention may also be made, in this category of polyalkylsiloxanes, of the products sold under the names ABIL WAX 9800 and ABIL WAX 9801 by Goldschmidt, which are poly(C₁-C₂₀)alkylsiloxanes.

The polyalkylarylsiloxanes are chosen, for example, from linear and/or branched polydimethyl(methylphenyl)siloxanes or polydimethyldiphenylsiloxanes with a viscosity ranging from 1×10⁻⁵ to 5×10⁻² m²/s at 25° C.

Mention may be made, among these polyalkylarylsiloxanes, by way of example, of the products sold under the following names:

SILBIONE oils of the 70 641 series from Rhône-Poulenc;

oils of the RHODORSIL 70 633 and 763 series from Rhône-Poulenc;

the oil DOW CORNING 556 COSMETIC GRADE FLUID from Dow Corning;

silicones of the PK series from Bayer, such as the product PK20;

silicones of the PN and PH series from Bayer, such as the products PN1000 and PH1000;

some oils of the SF series from General Electric, such as SF 1023, SF 1154, SF 1250 and SF 1265.

The silicone gums which can be present in the composition according to the disclosure may be, for example, polydiorganosiloxanes having high number-average molecular weights ranging from 200,000 to 1,000,000, used alone or as a mixture in at least one solvent. This at least one solvent can, for example, be chosen from volatile silicones, polydimethylsiloxane (PDMS) oils, polyphenylmethylsiloxane (PPMS) oils, isoparaffins, polyisobutylenes, methylene chloride, pentane, dodecane, tridecanes and mixtures thereof.

Exemplary mention may also be made of the following products:

polydimethylsiloxane gums,

polydimethylsiloxane/methylvinylsiloxane gums,

polydimethylsiloxane/diphenylsiloxane gums,

polydimethylsiloxane/phenylmethylsiloxane gums, and

polydimethylsiloxane/diphenylsiloxane/methylvinylsiloxane gums.

For example, products which may be used include the following mixtures:

mixtures formed from a polydimethylsiloxane hydroxylated at the chain end (named dimethiconol according to the nomenclature of the CTFA dictionary) and from a cyclic polydimethylsiloxane (named cyclomethicone according to the nomenclature of the CTFA dictionary), such as the product Q2 1401 sold by Dow Corning;

mixtures formed from a polydimethylsiloxane gum with a cyclic silicone, such as the product SF 1214 SILICONE FLUID from General Electric; this product is an SF 30 gum corresponding to a dimethicone, having a number-average molecular weight of 500,000, dissolved in the SF 1202 Silicone Fluid oil corresponding to decamethylcyclopentasiloxane;

mixtures of two PDMSs with different viscosities, for example of a PDMS gum and of a PDMS oil, such as the product SF 1236 from General Electric. The product SF 1236 is the mixture of an SE 30 gum defined above, having a viscosity of 20 m²/s, and of an SF 96 oil with a viscosity of 5.10⁻⁶ m²/s. In at least one embodiment, this product comprises 15% by weight relative to the total weight of the composition of SE 30 gum and 85% by weight relative to the total weight of the composition of an SF 96 oil.

For example, the organopolysiloxane resins optionally present in the composition according to the disclosure may be crosslinked siloxane systems including the units: R₂SiO_{2/2}, R₃SiO_{1/2}, RSiO_{3/2} and SiO_{4/2}, wherein R represents a hydrocarbon group possessing from 1 to 16 carbon atoms or a phenyl group. In at least one embodiment, these products are chosen from, those wherein R denotes a lower C₁-C₄ alkyl radical, for example chosen from methyl, and phenyl radicals.

Exemplary mention may be made, among these resins, of the product sold under the name DOW CORNING 593 or those sold under the names SILICONE FLUID SS 4230 and SILICONE FLUID SS 4267 by General Electric and which are silicones with a dimethyl/trimethylsiloxane structure.

Exemplary mention may also be made of resins of the trimethylsiloxysilicate type, sold, for example, under the names X22-4914, X21-5034 and X21-5037 by Shin-Etsu.

For example, the organomodified silicones optionally present in the composition according to the disclosure may be silicones as defined above which comprise, in their structure, at least one organofunctional groups attached via a hydrocarbon radical.

Exemplary mention may be made, among organomodified silicones, of the polyorganosiloxanes comprising:

polyethyleneoxy and/or polypropyleneoxy groups optionally comprising C₆-C₂₄ alkyl groups, such as the products known as dimethicone copolyol, sold by Dow Corning under the name DC 1248, or the SILWET L 722, L 7500, L 77 and L 711 oils from Union Carbide, and (C₁₂)alkyl methicone copolyol, sold by Dow Corning under the name Q2 5200;

thiol groups, such as the products sold under the names GP 72 A and GP 71 from Genesee;
alkoxylated groups, such as the product sold under the name SILICONE COPOLYMER F-755 by SWS SILICONES and ABIL WAX 2428, 2434 and 2440 by Goldschmidt;

hydroxylated groups, such as the polyorganosiloxanes possessing a hydroxyalkyl functional group described in French Patent Application Publication No. 2589476;

acyloxyalkyl groups, such as, for example, the polyorganosiloxanes described in U.S. Pat. No. 4,957,732;
anionic groups of the carboxyl type, such as, for example, in the products described in European Patent EP 186 507 from Chisso Corporation, or of the alkylcarboxyl type, such as those present in the product X-22-3701 E from Shin-Etsu; 2-hydroxyalkylsulphonate; 2-hydroxyalkylthiosuiphate, such as the products sold by Goldschmidt under the names ABIL S201 and ABIL 5255;

hydroxyacylamino groups, such as the polyorganosiloxanes described in European Application EP 342 834. Mention may be made, for example, of the product Q2-8413 from Dow Corning.

Exemplary mention may also be made, among organomodified silicones, of aminated silicones.

As used herein, the term “aminated silicone” is understood to mean any silicone comprising at least one primary, secondary or tertiary amine functional group or one quaternary ammonium group.

In at least one embodiment, the aminated silicones used in the cosmetic composition according to the present disclosure are chosen from:

(a) compounds corresponding to the following formula (I):

(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ (I)

wherein:

T is chosen from hydrogen atoms and phenyl, hydroxyl (—OH), C₁-C₈ alkyl, such as methyl, and C₁-C₈ alkoxy radicals, such as methoxy radicals,

a denotes an integer ranging from 0 to 3, such as 0,

b denotes 0 or 1, for example 1,

m and n are numbers such that the sum (n+m) can range, for example, from 1 to 2,000, such as from 50 to 150, it being possible for n to denote a number ranging from 0 to 1,999, such as from 49 to 149 and it being possible for m to denote a number from 1 to 2,000, for example from 1 to 10;

R¹ is a monovalent radical of —C_{q}H_{2q}L wherein q is a number ranging from 2 to 8 and

L is an aminated group, optionally quaternized, chosen from the groups:

—N(R²)—CH₂—CH₂—N(R²)₂;

—N(R²)₂; —N⁺(R²)₃Q⁻;

N⁺(R²)(H)₂Q⁻;

—N⁺(R²)₂HQ⁻; and

—N(R²)—CH₂—CH₂—N⁺(R²)(H)₂Q⁻,

wherein R² is chosen from hydrogen atoms, phenyls, benzyls and saturated monovalent hydrocarbon radicals, for example C₁-C₂₀ alkyl radicals, and Q⁻ represents a halide ion, such as, for example, fluoride, chloride, bromide or iodide.

In at least one embodiment, the aminated silicones corresponding to the definition of the formula (I) are chosen from the compounds corresponding to the following formula (II):

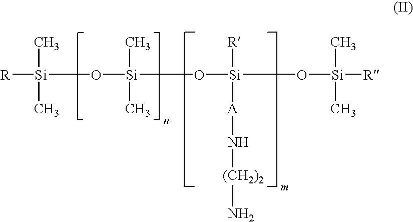

wherein R, R′ and R″, which are identical or different, are chosen from C₁-C₄ alkyl radicals, such as CH₃, C₁-C₄ alkoxy radicals, such as methoxy, and OH; A represents a linear or branched C₃-C₈, such as C₃-C₆, alkylene radical; m and n are integers depending on the molecular weight, the sum of which ranges from 1 to 2,000.

According to at least one embodiment, R, R′ and R″, which are identical or different, are chosen from C₁-C₄ alkyl and hydroxyl radicals, A represents a C₃ alkylene radical and m and n are such that the weight-average molecular weight of the compound ranges from 5,000 to 500,000. For example, compounds of this type are named “amodimethicone” in the CTFA dictionary.

According to another embodiment, R, R′ and R″, which are identical or different, are chosen from C₁-C₄ alkoxy or hydroxyl radicals, wherein at least one of the R or R″ radicals is an alkoxy radical and A represents a C₃ alkylene radical. In a further embodiment, the molar ratio of hydroxyl to alkoxy groups ranges from 0.2:1 to 0.4:1, and in at least one embodiment the ratio is equal to 0.3:1. Furthermore, m and n may be such that the weight-average molecular weight of the compound ranges from 2,000 to 10⁶. In at least one embodiment, n ranges from 0 to 999 and m ranges from 1 to 1,000, the sum of n and m ranging from 1 to 1,000.

Exemplary mention may be made, in this category of compounds, inter alia, of the product BELSIL®ADM 652 sold by Wacker.

According to yet another embodiment, R and R″, which are different, are chosen from C₁-C₄ alkoxy or hydroxyl radicals, wherein at least one of the R and R″ radicals is an alkoxy radical, R′ represents a methyl radical and A represents a C₃ alkylene radical. In a further embodiment, the molar ratio of hydroxyl to alkoxy groups ranges from 1:0.8 to 1:1.1, and in at least one embodiment is equal to 1:0.95. Furthermore, m and n are such that the weight-average molecular weight of the compound ranges from 2,000 to 200,000. In at least one embodiment, n ranges from 0 to 999 and m ranges from 1 to 1,000, the sum of n and m ranging from 1 to 1,000.

For example, mention may be made of the product FLUID WR® 1300 sold by Wacker.

According to another embodiment, R and R″ represent a hydroxyl radical, R′ represents a methyl radical and A is a C₄-C₈, for example C₄, alkylene radical. Furthermore, m and n are such that the weight-average molecular weight of the entity ranges from 2,000 to 10⁶ g/mol. In at least one embodiment, n ranges from 0 to 1,999 and m ranges from 1 to 2,000, the sum of n and m ranging from 1 to 2000.

For example, a product of this type is sold under the name DC28299 by Dow Corning.

It should be noted that the molecular weight of these silicones may be determined by gel permeation chromatography (for example at ambient temperature, using a polystyrene standard; with styragem μ columns; wherein the eluent is THF; at a flow rate of 1 mm/m; wherein 200 μl of a 0.5% by weight solution of silicone in THF are injected and the technique is carried out by refractometry and UV spectrometry).

In at least one embodiment, the product corresponding to the definition of formula (I) is the polymer named “trimethylsilylamodimethicone” in the CTFA dictionary, corresponding to the following formula (III):

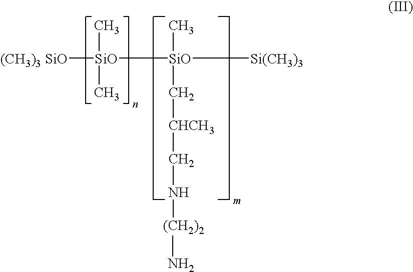

wherein n and m have the meanings given above in accordance with formula (I).

Such compounds are described, for example, in EP 95238; a compound of formula (III) is, for example, sold under the name O2-8220 by OSI.

(b) compounds corresponding to the following formula (IV):

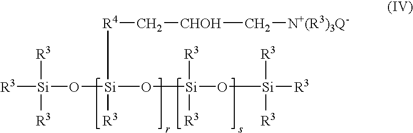

wherein:

R³ represents a monovalent C₁-C₁₈ hydrocarbon radical, such as a C₁-C₁₈ alkyl or C₂-C₁₈ alkenyl radical, for example methyl;

R⁴ represents a divalent hydrocarbon radical, such as a C₁-C₁₈ alkylene radical, or a divalent C₁-C₁₈, for example C₁-C₈, alkyleneoxy radical;

Q⁻ is a halide ion, such as chloride;

r represents a mean statistical value ranging from 2 to 20, for example from 2 to 8;

s represents a mean statistical value ranging from 20 to 200, for example from 20 to 50.

Such compounds are described, for example, in U.S. Pat. No. 4,185,087.

For example, a compound coming within this category is that sold by Union Carbide under the name UCAR SILICONE ALE 56.

c) quaternary ammonium silicones of formula (V):

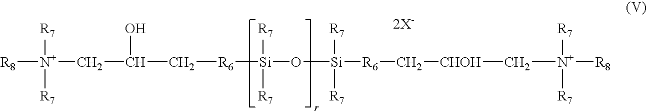

wherein:

R₇, which are identical or different, represent monovalent hydrocarbon radicals having from 1 to 18 carbon atoms, such as C₁-C₁₈ alkyl radicals, C₂-C₁₈ alkenyl radicals and rings comprising 5 or 6 carbon atoms, for example methyl;

R₆ represents divalent hydrocarbon radicals, such as C₁-C₁₈ alkylene radicals, or divalent C₁-C₁₈, for example C₁-C₈, alkyleneoxy radicals connected to Si via an SiC bonds;

R₈, which are identical or different, are chosen from hydrogen atoms, monovalent hydrocarbon radicals having from 1 to 18 carbon atoms, such as C₁-C₁₈ alkyl radicals and C₂-C₁₈ alkenyl radicals, and —R—NHCOR₇ radicals;

X⁻ is an anion, such as a halide ion, for example chloride, or an organic acid salt (such as acetate, and the like);

r represents a mean statistical value ranging from 2 to 200, for example from 5 to 100.

These silicones are, for example, described in Application EP-A-0 530 974.

d) aminated silicones of formula (VI):

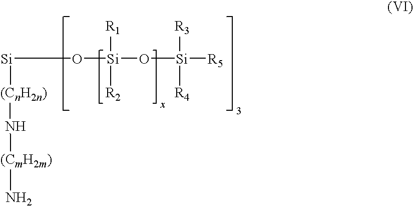

wherein:

R₁, R₂, R₃ and R₄, which are identical or different, are chosen from C₁-C₄ alkyl radicals and phenyl groups,

R₅ is chosen from C₁-C₄ alkyl radicals and hydroxyl groups,

n is an integer ranging from 1 to 5,

m is an integer ranging from 1 to 5,

and wherein x is chosen so that the amine number ranges from 0.01 to 1 meq/g.

In at least one embodiment, the silicones are the polysiloxanes comprising aminated groups, such as amodimethicones or trimethylsilylamodimethicones (CTFA, 4^{th} edition, 1997), and in at least one embodiment are silicones comprising quaternary ammonium groups.

When these compounds are employed, in at least one embodiment, they are jointly used with cationic and/or non-ionic surface-active agents.

Use may be made, by way of example, of the product sold under the name CATIONIC EMULSION DC 939 by Dow Corning, which comprises, in addition to the amodimethicone, a cationic surface-active agent which is trimethylcetylammonium chloride and a non-ionic surface-active agent of formula: C₁₃H₂₇—(OC₂H₄)₁₂—OH, known under the CTFA name TRIDECETH-12.

Another commercial product which can be used according to the disclosure is the product sold under the name Dow Corning Q2 7224 by Dow Corning, comprising, in combination, the trimethylsilylamodimethicone of formula (III) described above, a non-ionic surface-active agent of formula: C₈H—C₆H₄—(OCH₂CH₂)₄₀—OH, known under the CTFA name octoxynol-40, a second non-ionic surface-active agent of formula: C₁₂H₂₅—(OCH₂—CH₂)₆—OH, known under the CTFA name isolaureth-6, and propylene glycol.

For example, the silicone(s) are present in an amount ranging from 0 to 30% by weight relative to the total weight of the composition, for example from 0.2 to 20% by weight relative to the total weight of the composition.

For example, as explained above, the cosmetic composition according to the disclosure can also comprise at least one polymer other than the at least one lambda-carrageenan polysaccharide described above.

The at least one polymer other than the at least one lambda-carrageenan polysaccharide described above can be of vegetable or mineral natural origin and/or of synthetic origin.

As used herein, the term “polymer” is understood to mean a compound comprising the repetition of at least two units resulting from at least one compound referred to as monomer. This accordingly, for example, includes oligomers having a repeat number ranging from 2 to 10.

For example, the at least one polymer of natural origin can be chosen from pectins, celluloses, alginates, galactoarabinan, gum tragacanth, starches and sucrose.

The at least one polymer of vegetable origin modified by a synthetic route can be chosen, for example, from starch derivatives, such as carboxymethylstarch and distarch phosphate, and cellulose derivatives, such as hydroxyethylcellulose and carboxymethylcellulose.

The at least one polymer can be chosen from cationic, anionic, amphoteric and nonionic polymers.

As used herein, the term “cationic polymer” is understood to mean any polymer comprising cationic groups and/or groups which can be ionized to give cationic groups.

In one embodiment, the cationic polymers which can be used in the cosmetic composition according to the disclosure are chosen from polymers comprising primary, secondary, tertiary and/or quaternary amine groups, which form part of the polymer chain or are directly connected to the latter, and having a number-average molecular weight ranging from 500 to 5,000,000, for example from 1000 to 3,000,000.

Exemplary mention may be made, among these polymers, of the following cationic polymers:

(1) homopolymers or copolymers of acrylic or methacrylic esters or amides, possessing amino functional groups, comprising at least one unit of the following formulae:

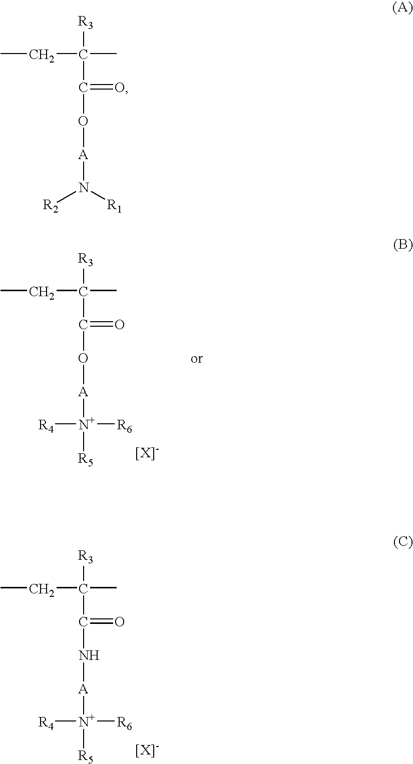

wherein:

R₁ and R₂, which are identical or different, each represent a hydrogen atom or an alkyl group having from 1 to 6 carbon atoms;

R₃ is chosen from hydrogen atoms and CH₃ groups;

A is a linear or branched alkyl group comprising from 1 to 6 carbon atoms or a hydroxyalkyl group comprising from 1 to 4 carbon atoms;

R₄, R₅ and R₆, which are identical or different, are chosen from alkyl groups having from 1 to 18 carbon atoms and benzyl groups;

X denotes a methosulphate anion or a halide, such as chloride or bromide.

The copolymers of family (1) additionally comprise at least one unit derived from comonomers which can be chosen from the family of acrylamides, methacrylamides, diacetone acrylamides, acrylamides and methacrylamides substituted on the nitrogen by lower (C₁₄) alkyl groups, groups derived from acrylic or methacrylic acids or from their esters, vinyllactams, such as vinylpyrrolidone and vinylcaprolactam, and vinyl esters.

Thus, exemplary mention may be made, among these copolymers of family (1), of:

copolymers of acrylamide and of dimethylaminoethyl methacrylate which are quaternized with dimethyl sulphate or with a dimethyl halide, such as that sold under the name HERCOFLOC® by Hercules,

copolymers of acrylamide and of methacryloyloxyethyltrimethylammonium chloride, described, for example, in Patent Application EP-A-080 976 and sold under the name BINAQUAT P 100 by Ciba-Geigy,

copolymers of acrylamide and of methacryloyloxyethyltrimethylammonium methosulphate, such as that sold under the name RETEN by Hercules,

vinylpyrrolidone/dialkylaminoalkyl acrylate or methacrylate copolymers, which may or may not be quaternized, such as the products sold under the name GAFQUAT® by ISP, such as, for example, GAFQUAT® 734 or GAFQUAT® 755, or the products named COPOLYMER® 845, 958 and 937. These polymers are, for example, described in detail in French Patents Nos. 2 077 143 and 2 393 573,

dimethylaminoethyl methacrylate/vinylcaprolactam/vinylpyrrolidone terpolymers, such as the product sold under the name GAFFIX® VC 713 by ISP, and vinylpyrrolidone/quaternized dimethylaminopropylmethacrylamide copolymers, such as the product sold under the name GAFQUAT® HS100 by ISP;

crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride or by copolymerization of acrylamide with dimethylaminoethyl methacrylate quaternized with methyl chloride, the homo- or copolymerization being followed by a crosslinking with a compound comprising olefinic unsaturation, such as methylenebisacrylamide. In at least one embodiment, use may be made of an acrylamide/methacryloyloxyethyltrimethylammonium chloride (20/80 by weight) crosslinked copolymer in the form of a dispersion comprising 50% by weight of the said copolymer in mineral oil. This dispersion is, for example, sold under the name of SALCARE® SC 92 by Ciba. In another embodiment, use may also be made of a crosslinked homopolymer of methacryloyloxyethyltrimethylammonium chloride comprising approximately 50% by weight of the homopolymer in mineral oil or in a liquid ester. These dispersions are, for example, sold under the names of SALCARE® SC 95 and SALCARE® SC 96 by Ciba.

(2) Cationic polysaccharides such as those chosen from:

a) cellulose ether derivatives comprising quaternary ammonium groups described in Patent FR 1 492 597. These polymers are also defined, for example, in the CTFA dictionary as quaternary ammoniums of hydroxyethylcellulose having reacted with an epoxide substituted by a trimethylammonium group.

b) cellulose derivatives grafted with a water-soluble monomer comprising a quaternary ammonium and described, for example, in U.S. Pat. No. 4,131,576, such as hydroxyalkylcelluloses, for example hydroxymethyl-, hydroxyethyl- or hydroxypropylcelluloses, such as those grafted with a methacryloyloxyethyltrimethylammonium, methacrylamidopropyltrimethylammonium or dimethyldiallylammonium salt.

For example, marketed products corresponding to this definition include the products sold under the names CELQUAT L 200 and CELQUAT H 100 by National Starch.

c) cationic polygalactomannans, such as those described in U.S. Pat. Nos. 3,589,578 and 4,031,307, such as guar gums comprising trialkylammonium cationic groups. Such products are sold, for example, under the trade names JAGUAR C13 S, JAGUAR C15 and JAGUAR C17 by Meyhall.

(3) quaternary copolymers of vinylpyrrolidone and of vinylimidazole.

(4) chitosans or their salts; in at least one embodiment the salts which can be used are chosen from chitosan acetate, lactate, glutamate, gluconate and pyrrolidonecarboxylate.

Exemplary mention may be made, among these compounds, of the chitosan having a degree of deacetylation of 90.5% by weight sold under the name KYTAN BRUT STANDARD by Aber Technologies or the chitosan pyrrolidonecarboxylate sold under the name KYTAMER® PC by Amerchol.

For example, the at least one anionic polymer generally used may include at least one polymer comprising groups derived from carboxylic, sulphonic or phosphoric acid and has a number-average molecular weight ranging from 500 to 5,000,000 g/mol.

For example, the carboxyl groups are contributed by unsaturated mono- or dicarboxylic acid monomers such as those corresponding to formula (VII):

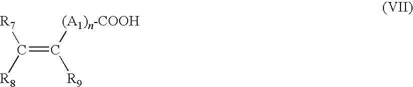

wherein n is an integer ranging from 0 to 10, A1 denotes a methylene group, optionally connected to the carbon atom of the unsaturated group or to the neighbouring methylene group when n is greater than 1 via a heteroatom, such as oxygen or sulphur, R7 is chosen from hydrogen atoms and phenyl and benzyl groups, R8 denotes a hydrogen atom or a lower alkyl or carboxyl group, and R9 is chosen from hydrogen atoms, lower alkyl groups and —CH2-COOH, phenyl and benzyl groups.

In at least one embodiment, in the abovementioned formula, a lower alkyl group denotes a group having 1 to 4 carbon atoms, such as methyl and ethyl groups.

For example, the at least one anionic polymer comprising carboxyl groups according to the disclosure is chosen from:

A) Homo- or copolymers of acrylic or methacrylic acid or their salts, such as the products sold under the names VERSICOL® E or K by Allied Colloid and ULTRAHOLD® by BASF, the copolymers of acrylic acid and of acrylamide sold in the form of their sodium salts under the names RETEN 421, 423 or 425 by Hercules or the sodium salts of polyhydroxycarboxylic acids.

B) Copolymers of acrylic or methacrylic acid with a monoethylenic monomer, such as ethylene, styrene, vinyl esters or esters of acrylic or methacrylic acid, optionally grafted onto a polyalkylene glycol, such as polyethylene glycol, and optionally crosslinked. Such polymers are described, for example, in French Patent No. 1 222 944 and German Application No. 2 330 956, the copolymers of this type comprising, in their chain, an optionally N-alkylated and/or -hydroxyalkylated acrylamide unit, such as described, for example in Luxembourgian Patent Applications Nos. 75370 and 75371 or provided under the name QUADRAMER by American Cyanamid. Exemplary mention may also be made of methacrylic acid/ethyl acrylate/tert-butyl acrylate terpolymers, such as the product sold under the name LUVIMER® 100 P by BASF.

Exemplary mention may also be made of the methacrylic acid/acrylic acid/ethyl acrylate/methyl methacrylate copolymers in aqueous dispersion sold under the name AMERHOLD® DR 25 by Amerchol.

C) Copolymers of crotonic acid, such as those comprising, in their chain, vinyl acetate or propionate units and optionally other monomers, such as allyl or methallyl esters, vinyl ether or vinyl ester of a linear or branched saturated carboxylic acid comprising a long hydrocarbon chain, such as those comprising at least 5 carbon atoms, it optionally being possible for these polymers to be grafted or crosslinked, or alternatively another monomer which is a vinyl, allyl or methallyl ester of an α- or β-cyclic carboxylic acid. Such polymers are described, inter alia, in French Patents Nos. 1 222 944, 1 580 545, 2 265 782, 2 265 781, 1 564 110 and 2 439 798. Commercial products coming within this class include, for example, the Resins 28-29-30, 26-13-14 and 28-13-10 sold by National Starch.

D) Copolymers of C₄-C₈ monounsaturated carboxylic acids or anhydrides chosen from:

copolymers comprising (i) at least one entity chosen from maleic, fumaric and itaconic acids and anhydrides and (ii) at least one monomer chosen from vinyl esters, vinyl ethers, vinyl halides, phenylvinyl derivatives, and acrylic acid and its esters, the anhydride functional groups of these copolymers optionally being monoesterified or monoamidated. Such polymers are described, for example, in U.S. Pat. Nos. 2,047,398, 2,723,248 and 2,102,113 and GB Patent No. 839 805. Such commercial products include, for example, those sold under the names GANTREZ® AN or ES by ISP;

copolymers comprising (i) at least one maleic, citraconic or itaconic anhydride unit and (ii) at least one monomer chosen from allyl or methallyl esters, optionally comprising at least one acrylamide, at least one methacrylamide, at least one α-olefin, at least one acrylic or methacrylic ester, at least one acrylic or methacrylic acid, or at least one vinylpyrrolidone group in its chain,

the anhydride functional groups of these copolymers optionally being monoesterified or monoamidated.

These polymers are, for example, described in French Patents Nos. 2 350 384 and 2 357 241.

E) Polyacrylamides comprising carboxylate groups.

The homopolymers and copolymers comprising sulpho groups are polymers comprising vinylsulphonic, styrenesulphonic, naphthalenesulphonic or acrylamidoalkylsulphonic units.

These polymers can, for example, be chosen from:

salts of polyvinylsulphonic acid having a molecular weight ranging from 1,000 to 100,000, as well as copolymers with an unsaturated comonomer, such as acrylic or methacrylic acids and their esters, as well as acrylamide or its derivatives, vinyl ethers and vinylpyrrolidone;

salts of polystyrenesulphonic acid, such as the sodium salts sold, for example, under the names FLEXAN® 500 and FLEXAN® 130 by National Starch. These compounds are described, for example, in Patent FR-A-2 198 719;

salts of polyacrylamidosulphonic acids, such as those mentioned in U.S. Pat. No. 4,128,631, such as the polyacrylamidoethylpropanesulphonic acid sold under the name COSMEDIA POLYMER HSP 1180 by Henkel.

For example, the anionic polymers are chosen from acrylic acid copolymers, such as the acrylic acid/ethyl acrylate/N-tert-butylacrylamide terpolymers sold, for example, under the name ULTRAHOLD® STRONG by BASF, copolymers derived from crotonic acid, such as the vinyl acetate/vinyl tert-butylbenzoate/crotonic acid terpolymers and the crotonic acid/vinyl acetate/vinyl neododecanoate terpolymers sold, for example, under the name RESIN 28-29-30 by National Starch, polymers derived from maleic, fumaric or itaconic acids or anhydrides with vinyl esters, vinyl ethers, vinyl halides, phenylvinyl derivatives, or acrylic acid and its esters, such as the monoesterified methyl vinyl ether/maleic anhydride copolymers sold, for example, under the name GANTREZ® by ISP, the copolymers of methacrylic acid and of methyl methacrylate sold under the name EUDRAGIT® L by Rohm Pharma, the copolymers of methacrylic acid and of ethyl acrylate sold under the name LUVIMER® MAEX or MAE by BASF, the vinyl acetate/crotonic acid copolymers sold, for example, under the name LUVISET CA 66 by BASF and the vinyl acetate/crotonic acid copolymers grafted by polyethylene glycol sold under the name ARISTOFLEX® A by BASF.

In at least one embodiment, the anionic polymers are chosen from monoesterified methyl vinyl ether/maleic anhydride copolymers sold under the name GANTREZ® ES 425 by ISP, acrylic acid/ethyl acrylate/N-tert-butylacrylamide terpolymers sold under the name ULTRAHOLD® STRONG by BASF, copolymers of methacrylic acid and of methyl methacrylate sold under the name EUDRAGIT® L by Rohm Pharma, vinyl acetate/vinyl tert-butylbenzoate/crotonic acid terpolymers and the crotonic acid/vinyl acetate/vinyl neododecanoate terpolymers sold under the name RESIN 28-29-30 by National Starch and copolymers of methacrylic acid and of ethyl acrylate sold under the name LUVIMER® MAEX or MAE by BASF.

For example, the amphoteric polymers which can be used in accordance with the disclosure can be chosen from polymers comprising B and C units distributed randomly in the polymer chain, wherein B denotes a unit deriving from a monomer comprising at least one basic nitrogen atom and C denotes a unit deriving from an acidic monomer comprising at least one carboxyl or sulpho group or else B and C can denote groups deriving from zwitterionic carboxybetaine or sulphobetaine monomers;

B and C can also denote, for example, a cationic polymer chain comprising primary, secondary, tertiary or quaternary amine groups, wherein at least one of the amine groups carries a carboxyl or sulpho group connected via a hydrocarbon group, or else B and C form part of a chain of a polymer comprising an α,β-dicarboxyethylene unit, one of the carboxyl groups of which has been reacted with a polyamine comprising at least one primary or secondary amine group.

In at least one embodiment, the amphoteric polymers corresponding to the definition given above are chosen from the following polymers:

(1) Copolymers comprising acidic vinyl units and comprising basic vinyl units, such as those resulting from the copolymerization of a monomer derived from a vinyl compound carrying a carboxyl group, such as acrylic acid, methacrylic acid, maleic acid and α-chloroacrylic acid, and of a basic monomer derived from a substituted vinyl compound comprising at least one basic atom, such as dialkylaminoalkyl methacrylate and acrylate and dialkylaminoalkylmethacrylamide and -acrylamide. Such compounds are described, for example, in U.S. Pat. No. 3,836,537.

(2) Polymers comprising units deriving:
a) from at least one monomer chosen from acrylamides and methacrylamides substituted on the nitrogen atom by an alkyl group,
b) from at least one acidic comonomer comprising at least one reactive carboxyl group, and
c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulphate.

In at least one embodiment, the N-substituted acrylamides or methacrylamides according to the disclosure are compounds wherein the alkyl groups comprise from 2 to 12 carbon atoms, and in at least one embodiment, are chosen from N-ethylacrylamide, N-tert-butylacrylamide, N-tert-octylacrylamide, N-octylacrylamide, N-decylacrylamide and N-dodecylacrylamide, and the corresponding methacrylamides.

In at least one embodiment, the at least one acidic comonomer is chosen from acrylic, methacrylic, crotonic, itaconic, maleic or fumaric acids and alkyl monoesters having 1 to 4 carbon atoms of maleic and fumaric acids and anhydrides.

In at least one embodiment, the at least one basic comonomer is chosen from aminoethyl, butylaminoethyl, N,N′-dimethylaminoethyl and N-tert-butylaminoethyl methacrylates.

In another embodiment, use is made of the copolymers for which the CTFA name (4th Ed., 1991) is Octylacrylamide/acrylates/butylaminoethyl methacrylate copolymer, such as the products sold under the name AMPHOMER® or LOVOCRYL® 47 by National Starch.

(3) Partially or completely acylated and crosslinked polyaminoamides deriving from polyaminoamides of general formula:

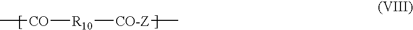

wherein R10 represents a divalent group derived from a saturated dicarboxylic acid, from an aliphatic mono- or dicarboxylic acid comprising an ethylenic double bond, from an ester of a lower alkanol having 1 to 6 carbon atoms of these acids, or from a group deriving from the addition of any one of these acids with a bisprimary or bissecondary amine, and Z denotes a group deriving from a bisprimary, mono- or bissecondary polyalkylenepolyamine and in at least one embodiment is chosen from:

a) the following group (IX) present in an amount ranging from 60 to 100 mol % relative to the total moles of the unit (VIII):

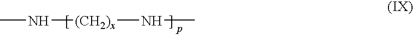

wherein x=2 and p=2 or 3, or else x=3 and p=2;

this group deriving from diethylenetriamine, triethylenetetraamine or dipropylenetriamine;

b) the above group (IX) present in an amount ranging from 0 to 40 mol % relative to the total moles of the unit (VIII), wherein x=2 and p=1 and which derives from ethylenediamine, or the group deriving from piperazine:

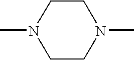

c) the group —NH—(CH2)6-NH— present in an amount ranging from 0 to 20 mol % relative to the total moles of the unit (VIII), deriving from hexamethylenediamine,

these polyaminoamides being crosslinked by addition reaction of at least one bifunctional crosslinking agent chosen from epihalohydrins, diepoxides, dianhydrides and bisunsaturated derivatives, by adding at least one crosslinking agent in an amount ranging from 0.025 to 0.35 mol per amine group of the polyaminoamide, and acylating by reacting with acrylic acid, chloroacetic acid or an alkanesultone or their salts.

In at least one embodiment, the saturated carboxylic acids are chosen from acids having from 6 to 10 carbon atoms, such as adipic, 2,2,4-trimethyladipic or 2,4,4-trimethyladipic, and terephthalic acids, and acids comprising an ethylenic double bond, such as, for example, acrylic, methacrylic and itaconic acids.

In another embodiment, the alkanesultones used in the acylation are chosen from propane- and butanesultones, and the salts of the acylating agents are chosen from sodium and potassium salts.

(4) Polymers comprising zwitterionic units of formula:

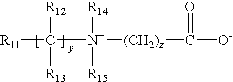

wherein R11 denotes a polymerizable unsaturated group, such as an acrylate, methacrylate, acrylamide or methacrylamide group, y and z represent an integer from 1 to 3, R12 and R13 represent a hydrogen atom or a methyl, ethyl or propyl group, and R14 and R15 represent a hydrogen atom or an alkyl group such that the sum of the carbon atoms in R14 and R15 does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Mention may be made, by way of example, of methyl methacrylate/methyl dimethylcarboxymethylammonioethylmethacrylate copolymers, such as the product sold under the name DIAFORMER Z301 by Sandoz.

(5) Polymers derived from chitosan comprising monomer units corresponding to the following formulae:

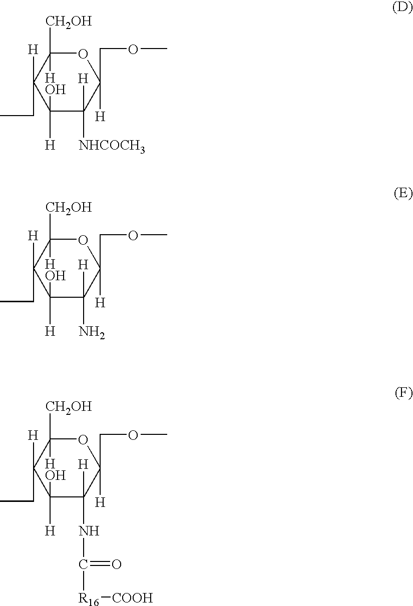

the unit (D) being present in an amount ranging from 0 to 30% relative to the total weight of the polymer, the unit (E) present in an amount ranging from 5 to 50% relative to the total weight of the polymer and the unit (F) present in an amount ranging from 30 to 90% relative to the total weight of the polymer, it being understood that, in this unit (F), R16 represents a group of formula:

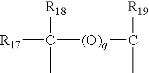

wherein, if q=0, R17, R18 and R19, which are identical or different, each represent a hydrogen atom, a methyl, hydroxyl, acetoxy or amino residue, a monoalkylamino residue or a dialkylamino residue, optionally interrupted by at least one nitrogen atom and/or optionally substituted by at least one amino, hydroxyl, carboxyl, alkylthio or sulpho group, or an alkylthio residue wherein the alkyl group carries an amino residue, at least one of the R17, R18 and R19 groups being, in this case, a hydrogen atom;

or, if q=1, R17, R18 and R19 each represent a hydrogen atom, and the salts formed by these compounds with bases or acids.

(6) Polymers comprising units corresponding to the general formula (X) are, for example, described in French Patent 1 400 366:

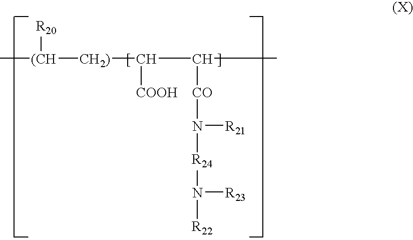

wherein R20 represents a hydrogen atom or a CH3O, CH3CH2O or phenyl group, R21 denotes a hydrogen atom or a lower alkyl group, such as methyl or ethyl, R22 denotes a hydrogen atom or a lower C1-C6 alkyl group, such as methyl or ethyl, and R23 denotes a lower C1-C6 alkyl group, such as methyl or ethyl, or a group corresponding to the formula: —R24-N(R22)2, R24 representing a —CH2-CH2-, —CH2-CH2-CH2- or —CH2-CH(CH3)- group and R22 having the meanings mentioned above.

(7) Polymers derived from the N-carboxyalkylation of chitosan, such as the N-(carboxymethyl)chitosan or the N-(carboxybutyl)chitosan sold under the name EVALSAN by Jan Dekker.

(8) Amphoteric polymers of the -D-X-D-X— type chosen from:
a) polymers obtained by reaction of chloroacetic acid or sodium chloroacetate with compounds comprising at least one unit of formula:

-D-X-D-X-D- (XI)

where D denotes a group:

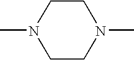

and X denotes the symbol E or E′, E or E′, which are identical or different, denoting a bivalent group which is a straight- or branched-chain alkylene group comprising up to 7 carbon atoms in the main chain which is unsubstituted or substituted by hydroxyl groups and which can additionally comprise oxygen, nitrogen or sulphur atoms or from 1 to 3 aromatic and/or heterocyclic rings; the oxygen, nitrogen and sulphur atoms being present in the form of ether, thioether, sulphoxide, sulphone, sulphonium, alkylamine or alkenylamine groups or hydroxyl, benzylamine, amine oxide, quaternary ammonium, amide, imide, alcohol, ester and/or urethane groups.
b) polymers of formula:

-D-X-D-X— (XII)

where D denotes a group:

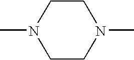

and X denotes the symbol E or E′ and at least once E′, E having the meaning indicated above and E′ being a bivalent group which is a straight- or branched-chain alkylene group having up to 7 carbon atoms in the main chain which is unsubstituted or substituted by at least one hydroxyl group and which comprises at least one nitrogen atom, the nitrogen atom being substituted by an alkyl chain optionally interrupted by an oxygen atom and necessarily comprising at least one carboxyl functional group and at least one hydroxyl functional group and betainized by reaction with chloroacetic acid or sodium chloroacetate.

(9) (C1-C5)Alkyl vinyl ether/maleic anhydride copolymers partially modified by semiamidation with an N,N-dialkylaminoalkylamine, such as N,N-dimethylaminopropylamine, or by semiesterification with an N,N-dialkylaminoalkanol. These copolymers can also comprise other vinyl comonomers, such as vinylcaprolactam.

Among the amphoteric polymers described above, in at least one embodiment according to the disclosure, the amphoteric polymers are chosen from those of family (3), such as the copolymers with the CTFA name of Octylacrylamideacrylates/butylaminoethyl methacrylate copolymer, such as the products sold under the names AMPHOMER®, AMPHOMER® LV 71 or LOVOCRYL® 47 by National Starch, and those of family (4), such as methyl methacrylate/methyl dimethylcarboxymethylammonioethylmethacrylate copolymers, sold, for example, under the name DIAFORMER® Z301 by Sandoz.

The non-ionic polymers which can be used according to the present disclosure are chosen, for example, from:

vinylpyrrolidone homopolymers;

polyalkyloxazolines;

vinyl acetate homopolymers;

vinyl acetate copolymers, such as, for example copolymers of vinyl acetate and of acrylic ester, copolymers of vinyl acetate and of ethylene, or copolymers of vinyl acetate and of maleic ester, for example of dibutyl maleate;

acrylic ester homopolymers and copolymers, such as, for example, copolymers of alkyl acrylates and of alkyl methacrylates, such as the products provided by Rohm & Haas under the names PRIMAL® AC-261 K and EUDRAGIT® NE 30 D, by BASF under the name 8845 or by Hoechst under the name APPRETAN® N9212;

copolymers of acrylonitrile and of a non-ionic monomer chosen, for example, from butadiene and alkyl(meth)acrylates; exemplary mention may be made of the products provided under the name CJ 0601 B by Rohm & Haas;

styrene homopolymers;

styrene copolymers, such as, for example, copolymers of styrene and of alkyl (meth)acrylate, such as the products MOWILITH® LDM 6911, MOWILITH® DM 611 and MOWILITH® LDM 6070 provided by Hoechst or the products RHODOPAS® SD 215 and RHODOPAS® DS 910 provided by Rhodia Chimie, copolymers of styrene, of alkyl methacrylate and of alkyl acrylate, copolymers of styrene and of butadiene or copolymers of styrene, of butadiene and of vinylpyridine;

polyamides;

vinyllactam homopolymers other than vinylpyrrolidone homopolymers, such as the polyvinylcaprolactam sold under the name LUVISKOL® Plus by BASF; and

vinyllactam copolymers, such as a poly(vinylpyrrolidone/vinyllactam) copolymer sold under the trade name LUVITEC® VPC 55K65W by BASF, poly(vinylpyrrolidone/vinyl acetate) copolymers, such as those sold under the name PVPVA® S630L by ISP or Luviskol® VA 73, VA 64, VA 55, VA 37 and VA 28 by BASF, and polyvinylpyrrolidone/vinyl acetate/vinyl propionate) terpolymers, such as, for example, that sold under the name Luviskol® VAP 343 by BASF.

In at least one embodiment, the alkyl groups of the non-ionic polymers mentioned above have from 1 to 6 carbon atoms.

For example, use may also be made, as polymers, of cationic, non-ionic, anionic or amphoteric polyurethanes which may or may not be functionalized and which may or may not be silicone-comprising, or their mixtures.

For example, the polyurethanes used in the present disclosure are those described in Applications EP 0 751 162, EP 0 637 600, EP 0 648 485 and FR 2 743 297, and in Applications EP 0 656 021 and WO 94/03510 of BASF and EP 0 619 111 of National Starch.

Exemplary mention may be made, as polyurethanes suitable in the present disclosure, of the products sold under the names LUVISET PUR® and LUVISET® Si PUR by BASF.

The at least one polymer other than the at least one lambda-carrageenan polysaccharide can also be chosen from associative or nonassociative synthetic thickening polymers. Exemplary mention may also be made of crosslinked acrylic acid homopolymers (INCI name: Carbomer) and nonionic or anionic associative polyurethanes, such as the ACULYN 44 and 46 and VISCOPHOBE DB 1000 compounds.

The polymer or polymers other than the at least one lambda-carrageenan polysaccharide described above is/are generally present in an amount ranging from 0 to 20% by weight relative to the total weight of the composition, for example from 0.2 to 10% by weight relative to the total weight of the composition.

As explained above, the composition according to the disclosure can comprise at least one surfactant.

The at least one surfactant optionally present in the composition according to the disclosure can be an anionic, amphoteric, nonionic, zwitterionic or cationic surfactant.

For example, the at least one surfactant suitable for the implementation of the present disclosure can be chosen from:

(i) Anionic Surfactants:

Non-limiting mention may be made, as examples of anionic surfactants which can be used, alone or as mixtures, in the context of the present disclosure, of the salts (such as alkali metal salts, for example sodium salts, ammonium salts, amine salts, aminoalcohol salts or magnesium salts) of the following compounds: alkyl sulphates, alkyl ether sulphates, alkylamido ether sulphates, alkylaryl polyether sulphates, monoglyceride sulphates; alkylsulphonates, alkyl phosphates, alkylamidesulphonates, alkylaryl-sulphonates, α-olefinsulphonates, paraffinsulphonates; (C₆-C₂₄)alkyl sulphosuccinates, (C₆-C₂₄)alkyl ether sulphosuccinates, (C₆-C₂₄)alkylamidesulphosuccinates; (C₆-C₂₄)alkyl sulphoacetates; (C₆-C₂Oacylsarcosinates and (C₆-C₂₄)acylglutamates. Use may also be made, for example, of (C₆-C₂₄)alkylpolyglycoside carboxylic esters, such as alkylglucoside citrates, alkylpolyglycoside tartrates and alkylpolyglycoside sulphosuccinates, alkyl sulpho-succinamates; acylisethionates and N-acyltaurates, the alkyl or acyl radical of all these different compounds, for example, comprising from 12 to 20 carbon atoms and the aryl radical, for example, denoting a phenyl or benzyl group. Non-limiting mention may also be made, among the anionic surfactants which can be used, of the salts of fatty acids, such as the salts of oleic, ricinoleic, palmitic and stearic acids, the acids of coconut oil or of hydrogenated coconut oil; and acyllactylates wherein the acyl radical comprises from 8 to 20 carbon atoms. Use may also be made, for example, of alkyl-D-galactosideuronic acids and their salts, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, such as those comprising from 2 to 50 alkylene oxide groups, for example ethylene oxide groups, and mixtures thereof.

(ii) Nonionic Surfactants:

The nonionic surface-active agents themselves are also compounds well known per se (in this respect see, for example, the “Handbook of Surfactants” by M. R. Porter, published by Blackie & Son (Glasgow and London), 1991, pp. 116-178). Thus, they can be chosen, by way of non-limiting examples, from polyethoxylated or polypropoxylated alcohols, α-diols or alkylphenols which have a fatty chain comprising, for example, from 8 to 18 carbon atoms, it being possible for the number of ethylene oxide or propylene oxide groups to range, for example, from 2 to 50. Exemplary mention may also be made of the copolymers of ethylene oxide and of propylene oxide and the condensates of ethylene oxide and of propylene oxide with fatty alcohols; the polyethoxylated fatty amides for example having from 2 to 30 mol of ethylene oxide, the polyglycerolated fatty amides comprising on average from 1 to 5 glycerol groups, such as from 1.5 to 4; the oxyethylenated sorbitan fatty acid esters having from 2 to 30 mol of ethylene oxide; the sucrose esters of fatty acids, the polyethylene glycol esters of fatty acids, alkylpolyglycosides, the N-alkylglucamine derivatives, or amine oxides, such as the (C₁₀-C₁₄)alkylamine oxides or the N-acylaminopropylmorpholine oxides. Exemplary mention may also be made of the alkylpolyglucosides.

Non-limiting mention may also be made of stearyl alcohol, cetearyl alcohol and Polysorbate 20.

Exemplary mention may be made, among the polyalkylglucosides, for example, of the products sold by Henkel under the name APG, such as the products APG 300, APG 350, APG 500, APG 550, APG 625 or APG base 10-12; the products sold by Seppic under the names TRITON CG 110 (or ORAMIX CG 110) and TRITON CG 312 (or ORAMIX NS 10); those sold by BASF under the name LUTENSOL GD 70; or those sold by Henkel under the names PLANTAREN 1200, PLANTAREN 1300 and PLANTAREN 2000 and PLANTACARE 2000, PLANTACARE 818 and PLANTACARE 1200.

(iii) Amphoteric or Zwitterionic Surfactants:

The amphoteric or zwitterionic surface-active agents, can by way of non-limiting examples, include derivatives of aliphatic secondary or tertiary amines wherein the aliphatic radical is a linear or branched chain comprising from 8 to 18 carbon atoms and comprising at least one water-solubilizing anionic group (for example, carboxylate, sulphonate, sulphate, phosphate or phosphonate); exemplary mention may also be made of (C₈-C₂₀)alkyl betaines, sulphobetaines, (C₈-C₂₀)alkyl amido(C₁-C₆)alkyl betaines and (C₈-C₂₀)alkyl amido(C₁-C₈)alkyl sulphobetaines.

Non-limiting mention may be made, among the amine derivatives, of the products sold under the MIRANOL name, as described in U.S. Pat. Nos. 2,528,378 and 2,781,354 and classified in the CTFA dictionary, 3rd Edition, 1982, under the names Amphocarboxyglycinates and Amphocarboxypropionates, with the respective structures:

R₂—CONHCH₂CH₂—N(R₃)(R₄)(CH₂COO—)

wherein: R₂ denotes a linear or branched C₅-C₂₀ alkyl radical originating, for example, from an acid R₂—COOH present in hydrolysed coconut oil, or a heptyl, nonyl or undecyl radical, R₃ denotes a β-hydroxyethyl group and R₄ denotes a carboxymethyl group; and

R₂′—CONHCH₂CH₂—N(B)(C)

wherein:

(B) represents —CH₂CH₂OX′, (C) represents —(CH₂)_{z}—Y′, with z equal to 1 or 2,

X′ denotes the —CH₂CH₂—COOH group or a hydrogen atom,

Y′ denotes —COOH or the —CH₂—CHOH—SO₃H radical,

R₂′ denotes a saturated or unsaturated and linear or branched C₅-C₂₀ alkyl radical of an acid R₉—COOH present, for example, in hydrolysed linseed oil or coconut oil, an alkyl radical, such as a C₇, C₉, C₁₁ or C₁₃ alkyl radical, a C₁₇ alkyl radical and its iso form, or an unsaturated C₁₇ radical.

These compounds are classified in the CTFA dictionary, 5th Edition, 1993, under the names Disodium Cocoamphodiacetate, Disodium Lauroamphodiacetate, Disodium Caprylamphodiacetate, Disodium Capryloamphodiacetate, Disodium Coco-amphodipropionate, Disodium Lauroamphodipropionate, Disodium Caprylampho-dipropionate, Disodium Capryloamphodipropionate, Lauroamphodipropionic acid and Cocoamphodipropionic acid.

Exemplary mention may be made, as example, of the cocoamphodiacetate sold under the trade name MIRANOL® C2M concentrate by Rhodia Chimie.

(iv) Cationic Surfactants:

Non-limiting mention may, for example, be made, among the cationic surfactants, of: the salts of optionally polyoxyalkylenated primary, secondary or tertiary fatty amines; quaternary ammonium salts, such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

Exemplary mention may also be made of the methyl(alkyl)(alkylamidoethyl)imidazolinium methosulphate (Quaternium-87) sold by Degussa under the reference VARISOFT W 575 PG, or the behenyltrimethylammonium chloride sold by Clariant under the reference GENAMIN KDMP. Mention may also be made, for example, of cetyltrimethylammonium chloride.

For example, use may also be made, as cationic surfactants, of quaternary ammonium salts comprising at least one ester functional group, such as those of following formula (XIII):

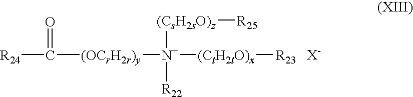

wherein:

R₂₂ is chosen from C₁-C₆ alkyl radicals and C₁-C₆ hydroxyalkyl or dihydroxyalkyl radicals;

R₂₃ is chosen from:

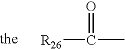

saturated or unsaturated, linear or branched, C₁-C₂ hydrocarbon radicals R₂₇,

the hydrogen atom;

R₂₅ is chosen from:

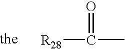

the saturated or unsaturated, linear or branched, C₁-C₆ hydrocarbon radicals R₂₉,

the hydrogen atom;

R₂₄, R₂₆ and R₂₈, which are identical or different, are chosen from saturated or unsaturated, linear or branched, C₇-C₂₁ hydrocarbon radicals;

r, s and t, which are identical or different, are integers having values ranging from 2 to 6;

y is an integer ranging having a value ranging from 1 to 10;

x and z, which are identical or different, are integers having values ranging from 0 to 10;

X⁻ is an organic or inorganic, simple or complex anion;

with the proviso that the sum x+y+z has a value ranging from 1 to 15, that, when x has the value 0, then R₂₃ denotes R₂₇ and that, when z has the value 0, then R₂₅ denotes R₂₉.

The R₂₂ alkyl radicals can be linear or branched and, in at least one embodiment are linear.

In at least one embodiment, R₂₂ denotes a methyl, ethyl, hydroxyethyl or dihydroxypropyl radical, such as a methyl or ethyl radical.

In another embodiment, the sum x+y+z has a value ranging from 1 to 10.

For example, when R₂₃ is an R₂ hydrocarbon radical, it can be long and have from 12 to 22 carbon atoms or short and have from 1 to 3 carbon atoms.

For example, when R₂₅ is an R₂₉ hydrocarbon radical, it may have from 1 to 3 carbon atoms.

In at least one embodiment, R₂₄, R₂₆ and R₂₈, which are identical or different, are chosen from saturated or unsaturated, linear or branched, C₁₁-C₂₁ hydrocarbon radicals, for example from saturated or unsaturated, linear or branched, C₁₁-C₂₁ alkyl and alkenyl radicals.

In another one embodiment, x and z, which are identical or different, have the value 0 or 1.

In at least one embodiment, y is equal to 1.

In a further embodiment, r, s and t, which are identical or different, have the value 2 or 3, and in at least one embodiment are equal to 2.

In one embodiment, the anion is chosen from halides (such as chlorides, bromides and iodides) and alkyl sulphates, such as methyl sulphates. However, in another embodiment, use may be made of methanesulphonate, phosphate, nitrate tosylate, an anion derived from an organic acid, such as acetate or lactate, or any other anion compatible with the ammonium comprising an ester functional group.

In at least one embodiment, the anion X⁻ is chosen from chloride and methyl sulphate.

In one embodiment, use is made, in the composition according to the disclosure, of the ammonium salts of formula (XIII) wherein:
R₂₂ denotes a methyl or ethyl radical,

x and y are equal to 1;

z is equal to 0 or 1;

r, s and t are equal to 2;

R₂₃ is chosen from:

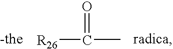

methyl, ethyl and C₁₄-C₂₂ hydrocarbon radicals, and

hydrogen atoms;

R₂₅ is chosen from:

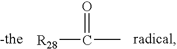

and

hydrogen atoms;

R₂₄, R₂₆ and R₂₈, which are identical or different, are chosen from saturated or unsaturated, linear or branched, C₁₃-C₁₇ hydrocarbon radicals, for example from saturated or unsaturated, linear or branched, C₁₃-C₁₇ alkyl and alkenyl radicals.

In at least one embodiment, the hydrocarbon radicals are linear.

Non-limiting mention may be made, for example, of the compounds of formula (XIII), such as diacyloxyethyldimethylammonium, diacyloxyethyl(hydroxyethyl)methyl-ammonium, monoacyloxyethyl(dihydroxyethyl)methylammonium, triacyloxy-ethyl(methyl)ammonium or monoacyloxyethyl(hydroxyethyl)dimethylammonium salts (such as chloride or methyl sulphate) and mixtures thereof. The acyl radicals, for example, may have 14 to 18 carbon atoms and may, for example, originate from a vegetable oil, such as palm oil or sunflower oil. When the compound comprises several acyl radicals, the latter can be identical or different.

These products are obtained, for example, by direct esterification of triethanolamine, of triisopropanolamine, of alkyldiethanolamine or of alkyldiisopropanol-amine, which are optionally oxyalkylenated, with fatty acids or with mixtures of fatty acids of vegetable or animal origin or by transesterification of their methyl esters. This esterification is followed by a quaternization using an alkylating agent, such as an alkyl (such as methyl or ethyl) halide, a dialkyl (such as methyl or ethyl) sulphate, methyl methanesulphonate, methyl para-toluenesulphonate, or glycol or glycerol chlorohydrin.

Such compounds are, for example, sold under the names DEHYQUART® by Henkel, STEPANQUAT® by Stepan, NOXAMIUM® by Ceca or REWOQUAT® WE 18 by Rewo-Witco.

In at least one embodiment, the composition according to the disclosure comprises a mixture of quaternary ammonium mono-, di- and triester salts, with a majority by weight of diester salts.

Use may be made, as mixture of ammonium salts, of, for example, the mixture comprising 15 to 30% by weight of acyloxyethyl(dihydroxyethyl)methyl-ammonium methyl sulphate, 45 to 60% of diacyloxyethyl(hydroxyethyl)methyl-ammonium methyl sulphate and 15 to 30% of triacyloxyethyl(methyl)ammonium methyl sulphate, the acyl radicals having from 14 to 18 carbon atoms and originating from optionally partially hydrogenated palm oil.

It is also possible to use the ammonium salts comprising at least one ester functional group described, for example, in U.S. Pat. Nos. 4,874,554 and 4,137,180.

In one embodiment, the at least one surfactant is present in a total amount ranging from 0 to 20% by weight relative to the total weight of the composition, such as from 0.05 to 10% by weight or from 0.1 to 5% by weight relative to the total weight of the composition.

As explained above, the composition according to the disclosure can comprise at least one fatty substance, for example of non-silicone nature, such as vegetable, animal, mineral and synthetic oils, fatty alcohols, fatty acids and waxes.

As used herein, the term “fatty alcohol” is understood to mean any saturated or unsaturated and linear or branched pure fatty alcohol comprising at least 8 carbon atoms.

The fatty alcohols can exhibit the structure R—OH, wherein R denotes a saturated or unsaturated and linear or branched radical comprising from 8 to 40 carbon atoms, such as from 8 to 30 and R, for example, denotes a C₁₂-C₂₄ alkyl or C₁₂-C₂₄ alkenyl group. For example, R can be substituted by at least one hydroxyl group.

Exemplary mention may be made, as examples of fatty alcohols, of lauryl, cetyl, dodecyl, decyl, stearyl, oleyl, behenyl, linoleyl, undecylenyl, palmitoleyl, arachidonyl and erucyl alcohol and mixtures thereof.

For example, the fatty alcohols can represent a mixture of fatty alcohols, which means that, in a commercial product, several types of fatty alcohols can coexist in the form of a mixture.

Exemplary mention may be made, as mixture of fatty alcohols, of cetylstearyl or cetearyl alcohol.

In one embodiment, at least one non-oxyalkylenated fatty alcohol is solid or pasty at a temperature of 25° C. As used herein, the term “fatty alcohol which is solid or pasty at 25° C.” is understood to mean, within the meaning of the present disclosure, a fatty alcohol exhibiting a viscosity, measured with a rheometer with a shear rate of 1 of greater than or equal to 1 Pa·s.

In at least one embodiment, the fatty alcohols used in the cosmetic composition according to the disclosure are chosen from cetyl alcohol and cetearyl alcohol.

As used herein, the term “fatty acids” is understood to mean any saturated or unsaturated and linear or branched pure carboxylic acid comprising at least 8 carbon atoms. Mention may be made, as examples of fatty acid, of lauric acid or oleic acid.

The cosmetic composition according to the disclosure can additionally comprise at least one cosmetic adjuvants chosen from fibers, fragrances, floral waters, essential oils, plasticizers, nonpolymeric polyols, sunscreens, basifying agents, acidifying agents, preservatives, permanent or temporary colorants, inorganic fillers, glitter, conditioning agents, antifoaming agents, moisturizing agents, humectants, emollients, nonpolymeric thickening agents, peptizing agents, ceramides, pseudoceramides, vitamins and provitamins, proteins, sequestering agents, solubilizing agents, oxidizing agents, corrosion inhibitors, reducing agents or antioxidants.

A person skilled in the art will take care to choose the possible adjuvants and their amounts so that they do not harm the properties of the composition according to the disclosure.

As used herein, the term “cosmetically acceptable medium” is understood to mean a medium compatible with keratinous substances, including the hair.

The cosmetically acceptable medium comprises water or a mixture of water and of at least one cosmetically acceptable solvent.

For example, the cosmetically acceptable solvent can be chosen from lower C₁-C₄ alcohols, from polyols, from polyol ethers, from C₅-C₁₀ alkanes, from C₃-C₄ ketones, from C₁-C₄ alkyl acetates, from dimethoxyethane, from diethoxyethane and from mixtures thereof.

For example, the composition according to the disclosure can be provided in the form of a cream or of a paste or, for example, in the form of a foam (aerosol or nonaerosol foam) or of a gel.

When the cosmetic composition according to the disclosure is provided in the form of a foam, it can be packaged in an aerosol. In this case, it comprises at least one propellant. The propellant or propellants are, for example, chosen from nonliquefied compressed gases, such as air, nitrogen, nitrous oxide, carbon dioxide or dimethylether, or else from liquefied gases, such as volatile hydrocarbons, for example n-butane, isobutane, propane, pentane and halogenated hydrocarbons. Use may be made, for example, of a mixture of at least one volatile hydrocarbon with dimethyl ether.

The foam can also be obtained without a propellant using a pump-action spray.

The composition according to the disclosure can also be a washing composition and can comprise a washing base known to a person skilled in the art, for example comprising at least one surfactant in an amount greater than 4%, for example an anionic surfactant.

The present disclosure also relates to a cosmetic treatment method comprising the application, to keratinous fibers, such as the hair, of a cosmetic composition as defined above.

The present disclosure still further relates to the use of a cosmetic composition for styling the hair.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLES

### Example 1

Cosmetic compositions according to the disclosure were prepared in the form of styling foams. Compositions 1 and 3 were delivered via an aerosol device and composition 2 was delivered via a foam pump device.

The formulations of compositions 1, 2 and 3 are given in Table 1. The contents are expressed by weight, with respect to the total weight of the composition.

TABLE 1

| | 1 | 2 | 3 |
|---|---|---|---|
| Lambda-Carrageenan | 0.1 to 2% | 1 to 10% | 1% |
| (SATIAGUM UTC 10- | | | |
| Degussa, WELGEENAN | | | |
| ED1039-Eurogum) | | | |
| Mg Li Na Laponite | 0.1 to 5% | 0.1 to 5% | 0.7% |
| (LAPONITE XLG- | | | |
| Degussa) | | | |
| Surfactant (Polysorbate-20, | 0.01 to 1% | | 1% |
| alkylpolyglycoside) | | | |
| Fixing polymer | | 1 to 10% | 1% |
| (polyvinylpyrrolidone) | | | |
| Mono- or polysaccharide | 0.1 to 10% | | 3% |
| (honey, sucrose) | | | |
| Starch derivative (potato | | 0.1 to 10% | 1.5% |
| maltodextrin) | | | |
| Denatured alcohol | 0 to 20% | 0 to 20% | |
| Preservatives, neutralizing | q.s. | q.s. | q.s. |
| agent, fragrance | | | |
| Propellent gas | 2 to 10% | | 5% |
| (hydrocarbons, CO₂, | | | |
| nitrous oxide, and the | | | |
| like) | | | |
| Water | q.s. for 100 | q.s. for 100 | q.s. for 100 |

Compositions 1 to 3 conferred good styling properties on the hair while preventing the formation of residues when a comb was passed through the hair.

### Example 2

Cosmetic compositions according to the disclosure were prepared in the form of styling gels.

The formulations of compositions 4, 5 and 6 are given in Table 2. The contents are expressed by weight, with respect to the total weight of the composition.

TABLE 2

| | 4 | 5 | 6 |
|---|---|---|---|
| Lambda-Carrageenan | 1 to 10% | 1 to 10% | 2% |
| (SATIAGUM UTC 10- | | | |
| Degussa, | | | |
| WELGEENAN | | | |
| ED1039-Eurogum) | | | |
| Mg Li Na Laponite | 0.1 to 5% | 0.1 to 5% | 5% |
| (LAPONITE XLG-Degussa) | | | |
| Mono- or polysaccharide | 0.1 to 10% | | 3% |
| (honey, sucrose) | | | |
| Starch derivative (potato | | 0.1 to 10% | 5% |
| maltodextrin) | | | |
| Surfactant (Polysorbate-20, | | 0.1 to 1% | 0.5% |
| alkylpolyglucoside) | | | |
| Fixing polymer | | 1 to 10% | 1% |
| (polyvinylpyrrolidone) | | | |
| Denatured alcohol | 0 to 20% | 0 to 20% | 2% |
| Silicone (dimethicone, | 0 to 20% | | |
| dimethiconol) | | | |
| Cations (behenyl- | 0 to 2% | | 2% |
| trimethylammonium | | | |
| chloride, cetyl- | | | |
| trimethylammonium chloride) | | | |
| Preservatives, neutralizing | q.s. | q.s. | q.s. |
| agent, fragrance | | | |
| Water | q.s. for 100 | q.s. for 100 | q.s. for 100 |

Compositions 4 to 6 conferred good styling properties on the hair while preventing the formation of residues when a comb was passed through the hair.

What is claimed is:

1. A cosmetic composition comprising, in a cosmetically acceptable medium:
at least one lambda-carageenan polysaccharide, and
inorganic particles chosen from particles comprising at least 10% by weight of at least one silicate.

2. The cosmetic composition according to claim 1, wherein the molecular weight (MW) of the at least one lambda-carageenan polysaccharide ranges from 100,000 to 1,000,000 g/mol.

3. The cosmetic composition according to claim 1, wherein the at least one lambda-carageenan polysaccharide is present in an amount ranging from 0.01 to 30% by weight relative to the total weight of the composition.

4. The cosmetic composition according to claim 1, wherein the inorganic particles are chosen from particles of at least one silicate.

5. The cosmetic composition according to claim 1, wherein the at least one silicate is chosen from magnesium, lithium, and sodium silicates.

6. The cosmetic composition according to claim 1, wherein the inorganic particles comprising at least 10% by weight of at least one silicate are present in an amount ranging from 0.01 to 20% by weight relative to the total weight of the composition.

7. The cosmetic composition according to claim 1, wherein the ratio of the total weight of the at least one lambda-carrageenan polysaccharide(s) to the total weight of inorganic particles comprising at least one silicate an amount of at least 10% by weight relative to the total weight of the particles has a value that ranges from 0.1 to 10.

8. The cosmetic composition according to claim 1, further comprising at least one additive chosen from silicones, fatty substances, polymers other than the at least one lambda-carrageenan polysaccharide and surfactants.

9. The cosmetic composition according to claim 1, further comprising at least one cosmetic adjuvant chosen from fibers, fragrances, floral waters, essential oils, plasticizers, nonpolymeric polyols, sunscreens, basifying agents, acidifying agents, preservatives, permanent colorants, temporary colorants, inorganic fillers, glitter, conditioning agents, antifoaming agents, moisturizing agents, humectants, emollients, nonpolymeric thickening agents, peptizing agents, ceramides, pseudoceramides, vitamins, provitamins, proteins, sequestering agents, solubilizing agents, oxidizing agents, corrosion inhibitors, reducing agents and antioxidants.

10. The cosmetic composition according to claim 1, wherein the cosmetically acceptable medium comprises water or a mixture of water and of at least one other cosmetically acceptable solvent.

11. The cosmetic composition according to claim 10, wherein the at least one other cosmetically acceptable solvent is chosen from lower C₁-C₄ alcohols, polyols, polyol ethers, C₅-C₁₀ alkanes, C₃-C₄ ketones, C₁-C₄ alkyl acetates, dimethoxyethane, and diethoxyethane.

12. The cosmetic composition according to claim 1, wherein the composition is provided in a form chosen from aerosols, nonaerosol foams and gels.

13. The cosmetic composition according to claim 1, further comprising at least one washing base.

14. A cosmetic treatment method, comprising applying to keratinous fibers a cosmetic composition comprising, in a cosmetically acceptable medium,
at least one lambda-carageenan polysaccharide, and
inorganic particles chosen from particles comprising at least 10% by weight of at least one silicate.

15. The cosmetic treatment method of claim 14, further comprising styling the hair.

16. The cosmetic composition according to claim 13, wherein the at least one washing base comprises at least one surfactant in a total amount of more than 4% by weight relative to the total weight of the composition.

17. The cosmetic composition according to claim 16, wherein the at least one surfactant is an anionic surfactant.

18. The cosmetic composition according to claim 2, wherein the molecular weight (MW) of the at least one lambda-carageenan polysaccharide ranges from 250,000 to 800,000 g/mol.

19. The cosmetic composition according to claim 3, wherein the at least one lambda-carageenan polysaccharide is present in a total amount ranging from 1 to 10% by weight relative to the total weight of the composition.

20. The cosmetic composition according to claim 6, wherein the inorganic particles comprising at least 10% by weight of at least one silicate are present in an amount ranging from 0.1 to 5% by weight relative to the total weight of the composition.
